(19) United States
(12) Patent Application Publication
(10) Pub. No.: US 2017/0300660 A1
(43) Pub. Date: Oct. 19, 2017

(54) METHOD AND APPARATUS FOR TRACKING MOVEMENT OF AN OBJECT WITHIN A CONTAINER

(71) Applicant: LuminX Corporation, Rancho Santa Fe, CA (US)

(72) Inventors: Noam A. Ziv, San Diego, CA (US); Jonathan Taub, san Diego, CA (US); Donald Jones, San Diego, CA (US); Maya Ziv, San Diego, CA (US)

(21) Appl. No.: 15/490,835

(22) Filed: Apr. 18, 2017

Related U.S. Application Data
(60) Provisional application No. 62/323,794, filed on Apr. 18, 2016.

(51) Int. Cl. G06F 19/00; A61J 1/03; A61J 7/04; H04L 29/06; G06Q 10/08

(52) U.S. Cl. CPC G06F 19/3462; H04L 63/0435; G06Q 10/087; A61J 7/0418; A61J 7/0481; A61J 1/03; G06Q 2220/10

(57) ABSTRACT

Certain aspects of the present disclosure provide a platform for monitoring patient adherence to a medication regimen. Certain aspects of the present disclosure also proved various components that may help enable such a platform, such as a smart collar capable of detecting removal and/or insertion of objects from a container (such as a pill container).

### CROSS-REFERENCE TO RELATED APPLICATIONS

This Application claims priority to 1) U.S. Provisional Application No. 62/323,794 entitled “SMART COLLAR FOR TRACKING PATIENT ADHERENCE” filed Apr. 18, 2016, the disclosure of which is hereby expressly incorporated in its entirety by reference herein.

### BACKGROUND

### Field of the Disclosure

Certain aspects of the present disclosure generally relate to healthcare and, more particularly, to mechanisms for monitoring and/or promoting patient adherence to a prescribed regimen.

### Description of Related Art

Patient adherence generally refers to how well a patient takes medication, according to a regimen prescribed by a healthcare provider. In other words, patients are considered adherent when they take the prescribed medications at doses and times according to the regimen. Patient adherence is obvious a key component of treatment success. Non-adherence can lead to poor patient outcome and increased health costs.

As patient non-adherence has been recognized a contributing factor in rising health care costs, recent efforts have been made to monitor patient adherence. Such monitoring has a potential to help reduce cost and improve patient health by improving the effectiveness of care delivered. Such monitoring may help distinguish between poor treatment response and patient non-adherence. As such, monitoring adherence may help guide providers in prescribing decisions, identifying causes of non-adherence, and take measures to promote adherence.

Conventional mechanisms for monitoring adherence include clinical assessments, where patient usage is observed and recorded, and patient self-reporting. Each of these mechanisms has their own drawbacks. For example, clinical assessments of patient adherence are typically inconvenient, costly, and time-consuming, while patient self-reporting is notoriously unreliable.

For these reasons, improved mechanisms for monitoring and/or promoting patient adherence are desirable.

### SUMMARY

The systems, methods, and devices of the disclosure each have several aspects, no single one of which is solely responsible for its desirable attributes. Without limiting the scope of this disclosure as expressed by the claims, which follow, some features will now be discussed briefly. After considering this discussion, and particularly after reading the section entitled “Detailed Description” one will understand how the features of this disclosure provide advantages that include improved communications in a wireless network.

Aspects of the present disclosure provide an apparatus for tracking a count of objects in a container. The apparatus generally includes a housing mountable at an opening of the container, at least one detector proximate the opening and responsive to removal of objects from (or insertion of objects into) the container, a processor configured to process a first signal generated by the detector to update a monitored count of objects in the container, and an interface for transmitting a wireless signal indicative of at least one of the monitored count or a change in the monitored count.

Aspects of the present disclosure provide an apparatus for monitoring patient adherence to a medication regimen. The apparatus generally includes a container for holding objects, a housing mountable at an opening of the container, at least one detector proximate the opening and responsive to removal of objects from the container, a processor configured to process a first signal generated by the detector to update a monitored count of objects in the container, and an interface for transmitting a wireless signal indicative of at least one of the monitored count or a change in the monitored count.

Certain aspects of the present disclosure also provide various methods, apparatus, and computer-program products for performing operations performed by the apparatus described above.

An aspect of the present disclosure provides an apparatus for tracking movement of an object within a container. The apparatus comprises a sensor circuit, a hardware processor, and a transmitter circuit. The sensor circuit is configured to detect at least one of a removal of the object from or insertion of the object into a container. The sensor circuit is also configured to generate a signal in response to detecting the removal of the object from or insertion of the object into the container. The hardware processor is configured to determine a time at which the object was removed from or inserted into the container based on the signal generated by the sensor circuit. The hardware processor is also configured to generate a message indicating that the object was removed from or inserted into the container at the determined time. The hardware processor is further configured to select an encryption key from a memory circuit storing a library of encryption keys and encrypt the generated message for transmission to a monitoring device according to the selected encryption key. The transmitter circuit is configured to wirelessly transmit the encrypted message to the monitoring device.

Another aspect of the present description provides a method for tracking movement of an object within a container. The method comprises detecting at least one of a removal of the object from or insertion of the object into a container and generating a signal in response to detecting the removal of the object from or insertion of the object into the container. The method also comprises determining a time at which the object was removed from or inserted into the container based on the signal generated by the sensor circuit. The method further comprises generating a message indicating that the object was removed from or inserted into the container at the determined time. The method also further comprises selecting an encryption key from a memory circuit storing a library of encryption keys and encrypting the generated message for transmission to a monitoring device according to the selected encryption key. The method further also comprises wirelessly transmitting the encrypted message to the monitoring device.

An aspect of the present disclosure provides an apparatus for tracking movement of an object within a container. The apparatus comprises means for detecting at least one of a removal of the object from or insertion of the object into a container and means for generating a signal in response to detecting the removal of the object from or insertion of the object into the container. The apparatus further comprises means for determining a time at which the object was removed from or inserted into the container based on the signal generated by the sensor circuit and means for generating a message indicating that the object was removed from or inserted into the container at the determined time. The apparatus also comprises means for selecting an encryption key from a memory circuit storing a library of encryption keys and means for encrypting the generated message for transmission to a monitoring device according to the selected encryption key. The apparatus further also comprises means for wirelessly transmitting the encrypted message to the monitoring device.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 illustrates a diagram of an example adherence-monitoring platform, in accordance with certain aspects of the present disclosure.

FIG. 2 illustrates a diagram of an example adherence-monitoring platform, in accordance with certain aspects of the present disclosure.

FIG. 3 illustrates example operations for monitoring object removal (and/or insertion) from a container, in accordance with certain aspects of the present disclosure.

FIG. 4 illustrates example operations for updating adherence information, in accordance with certain aspects of the present disclosure.

FIG. 5A illustrates an example sequence of operations for updating adherence information via a container with a smart collar, in accordance with certain aspects of the present disclosure.

FIG. 5B illustrates an example sequence of operations for communicating an alert based on adherence information via a container with a smart collar, in accordance with certain aspects of the present disclosure.

FIG. 6 illustrates an example container with a smart collar for monitoring removal and/or insertion of items from the container, in accordance with certain aspects of the present disclosure.

FIG. 7A illustrates an example smart collar with one or more sensors to detect travel of an object (such as a pill) from an opening of the smart collar, in accordance with certain aspects of the present disclosure.

FIG. 7B illustrates an example arrangement of one or more sensors, in accordance with certain aspects of the present disclosure.

FIG. 7C illustrates another example arrangement of one or more sensors, in accordance with certain aspects of the present disclosure.

FIG. 8 illustrates an example schematic diagram of a smart collar, in accordance with aspects of the present disclosure.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Aspects of the present disclosure generally relate to a platform for monitoring patient adherence to a medication regimen. Aspects of the present disclosure also relate to various components that may help enable such a platform, such as a smart collar that can detect and communicate information related to adherence. As described herein, a smart collar that seamlessly integrates with a medicine container may result in a cost effective mechanism to communicate accurate (e.g., on the order of per-pill accuracy) information regarding patient adherence.

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method, which is practiced using other structure, functionality, or structure and functionality in addition to or other than the various aspects of the disclosure set forth herein. It should be understood that any aspect of the disclosure disclosed herein may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different wireless technologies, system configurations, networks, and transmission protocols, some of which are illustrated by way of example in the figures and in the following description of the preferred aspects. The detailed description and drawings are merely illustrative of the disclosure rather than limiting, the scope of the disclosure being defined by the appended claims and equivalents thereof.

### An Example Platform for Monitoring Patient Adherence

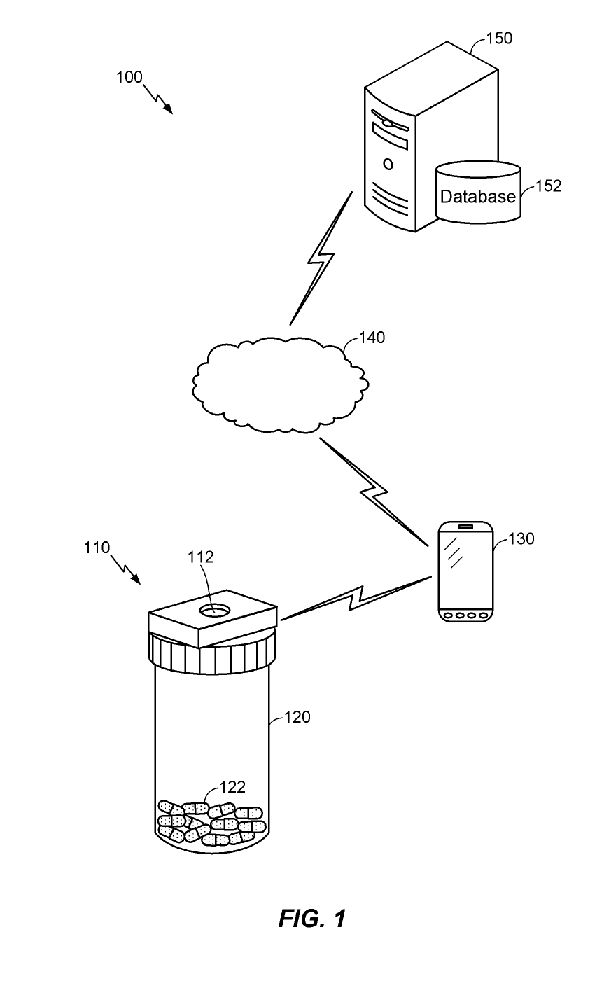

FIG. 1 illustrates a system 100 in which aspects of the disclosure may be performed. The system 100 may be considered a could-based platform for monitoring patient adherence. The system 100 includes a server 150 with a database 152, a network 140, a smartphone 130, and a medication bottle or container 120. The container 120 includes a plurality of objects 122 (e.g., pills or medication). The container 120 may be a cylinder that is enclosed at one end and open at the other end. The open end of the container 120 may be referred to herein as the “mouth” of the container 120. The container 120 may include a smart collar 110 coupled to the mouth of the container 120. The smart collar 110 may include an opening 112 that passes through the entire smart collar 110 and provides for the dispensing of the objects 122 from or inserting of the objects 122 into the container 120.

In the illustrated example, the smart collar 110 may be fitted to the container 120 and configured to monitor the removal and/or insertion of the objects 122 from the container 120. As will be described in greater detail below, the smart collar 110 may include one or more components (such as photo-interrupter or processor) capable of detecting and tracking the removal and/or insertion of the objects 122 (or any detectable amount of container contents) through the opening 112.

As an illustrative, but not limiting example, the container 120 may be a pill bottle and the objects 122 may be pills. Thus, the smart collar 110 may maintain and update a pill count as pills are taken from or added to the container 120. In such cases, the smart collar 110 may be adapted to couple with standard size pill bottles (e.g., replacing standard lids or being fitted into the standard size pill bottles so existing lids may continue to be used) or with custom size pill bottles. The smart collar 110 may be a separate component or, in some cases, may be an integral part of the container 120. While the pill bottle is described to facilitate understanding, the techniques and mechanisms described herein may be used to monitor any type of detectable content (e.g., whether solid, liquid, or gas) removed from or added to the container 120.

The smart collar 110 may have one or more interfaces allowing communication with the network 140, such as a cloud-computing network. As used herein, the term cloud computing generally refers to any type of network-based (e.g., Internet-based) computing that provides shared processing resources and data to devices on demand. As such, cloud computing may be considered a model for enabling on-demand access to a shared pool of configurable computing resources, which can be rapidly provisioned and released with minimal management effort.

The network may be used to exchange messages among several interacting spatially separated devices. In some embodiments, the network may be classified according to geographic scope, which could be, for example, a metropolitan area, a local area, or a personal area. Such a network may be designated respectively as a wide area network (WAN), metropolitan area network (MAN), local area network (LAN), wireless local area network (WLAN), or personal area network (PAN). Wireless communication networks may be widely deployed to provide various types of communication content such as voice and data. Typical wireless communication networks may be capable of supporting communication with multiple users by sharing available network resources (e.g., bandwidth, transmit power). Examples of such networks may include code division multiple access (CDMA) networks, time division multiple access (TDMA) networks, frequency division multiple access (FDMA) networks, orthogonal frequency division multiple access (OFDMA) networks, and the like. Additionally, the networks can conform to specifications such as third generation partnership project (3GPP), 3GPP2, 3GPP long-term evolution (LTE), LTE Advanced (LTE-A), LTE Unlicensed (LTE-U), LTE Direct (LTE-D), License-Assisted Access (LAA), MuLTEfire, etc. These networks may be accessed by various types of user equipment (stations) adapted to facilitate wireless communications, where multiple stations share the available network resources (e.g., time, frequency, and power).

Communications between the network 140 and the collar 110 may be bi-directional and may be used to exchange event information, configuration, alerts, or other information. In some embodiments, communications between the network 140 and the smart collar 110 may be unidirectional (e.g., from the smart collar 110 to the network 140) and communications to the patient using the smart collar 110 may occur via the smartphone 130.

In general, smart collar 110 to network 140 communications may be opportunistic and may occur via one or more paths, over multiple types of links (possibly wireless), either directly or indirectly (via gateway). In some cases, a device other than the smartphone 130 may serve as a gateway to the network 140. For example, gateway functionality may be provided as part of another connected device (TV, thermostat, Echo, or the like, not shown in this figure).

In the present example, the network 140 may allow information from the smart collar 110 to be communicated (e.g., via packets or other type messages) to the server 150 that monitors patient adherence. Information related to a pill count may include, for example, an absolute or relative pill count, change in pill count, number or rate of pills taken over a given time period, times at which pills were refilled, etc. Such information related to one or more patients may be stored in the database 152 and such information may be analyzed as part of an overall scheme to monitor and attempt to encourage patient adherence to a medication regimen.

The smart collar 110 may include any combination of interfaces to communicate with network 140 directly or indirectly. In the example illustrated in FIG. 1, the smart collar 110 may indirectly communicate with the network 140 via smartphone 130 (such as a mobile device), for example, via Bluetooth, Bluetooth low energy (LE), ZigBee, Wi-Fi, NFC (or other type of wireless local area network-WLAN). The smartphone may run an application (app) that is configured to communicate with the smart collar 110, gather data and, in some cases, present adherence data to a patient and/or notifications to a patient.

As will be described in greater detail below, in some cases, such an app may also be used to view information regarding a medicine as an alternative (or in addition) to conventional printed (and sometimes difficult to read) documentation.

In some cases, the smart collar 110 may include local storage to store information such as a medication regimen, the information for a particular medication, or other information. In some embodiments, the medication regimen may include a schedule or frequency at which medication (e.g., the medication included in the container 120) is to be dispensed from the container 120 and consumed by the patient. In some embodiments, the medication regimen may include information regarding a plurality or all medications that the patient consumes, both prescribed and over-the-counter medications. In some embodiments, the medication regimen may include more than just medications but also other activities or procedures that may affect the patient's health, such as physical activity or weekly dialysis, etc.

Once provisioned with the medication regimen information, local storage of the smart collar 110 may provide for autonomous collar operation when the network is not accessible.

In some cases, the cloud-computing network 140 may update information stored on the smart collar 110 such as medication regimen, the information for a particular medication (“booklet”), or other information. The smart collar 110 described herein may accommodate such updates at home (e.g. with no need to visit a pharmacy), or at other locations (pharmacy or medical provider), or at remote locations (e.g., while traveling, while at work, etc.).

In some cases, in order to protect sensitive patient information, the smart collar 110 may encrypt events, the medication regimen, and other provisioned or collected information. Such encrypted information may be saved locally and may be deleted upon server command or remote request by one of the patient or a healthcare provider.

In some cases, devices other than the collar (e.g., apps on phones or TVs, laptops, security systems, thermostats, etc.) may be used to present informational content to the patient or person associated with the patient. Depending on the deployment, such information may or may not follow the same communication path as the server to collar communications. In some cases, once activated and provisioned, a collar may operate autonomously (e.g., without any server communication) and, in such cases, the collar may generate local informational content (e.g., using whatever capabilities it has) as well as collect and save event information (e.g., any suitable form of local memory). In some cases, such local alerts/storage may be performed only until a connection is obtained. In other cases, such local alerts/storage may be supplemental (e.g., performed regardless of connectivity).

As noted herein, certain medication (“booklet”) information may be stored locally to a collar. In such cases, such information may be accessed by connecting a viewer to the collar (app, TV, computer, refrigerator, or the likes).

In some cases, a collar may be provisioned to help users locate (“find”) it. In some cases, a collar may include other “user facing” features such as an “OK reminder” (e.g., indicating to a user they are in compliance and taking pills on time).

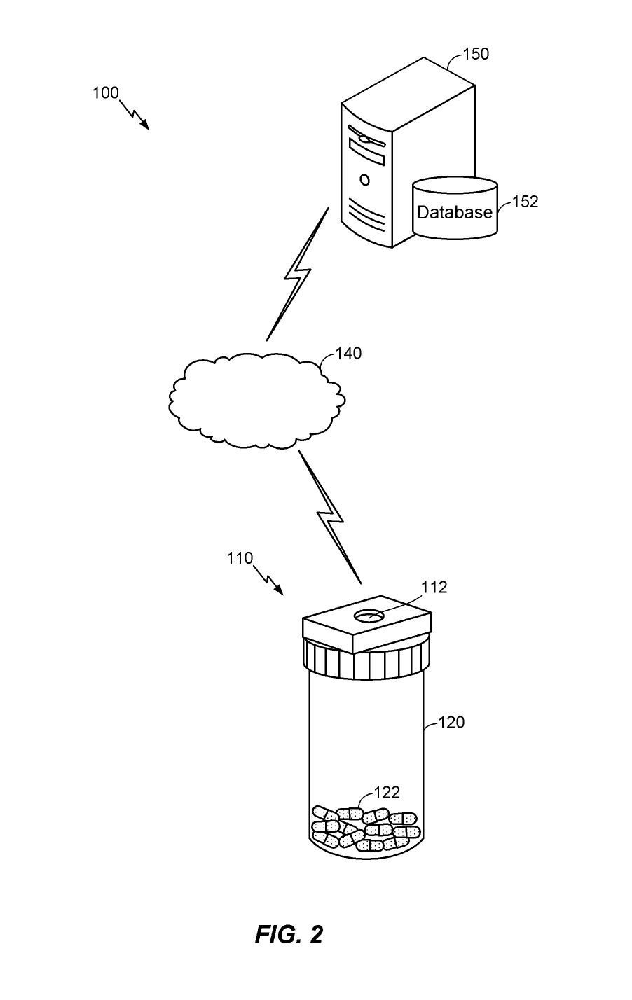

As illustrated in FIG. 2, in some cases, a smart collar may communicate via different types of connections, such as directly with the network 140 (e.g., without needing the smartphone 130 or any other separate device for connectivity to the network 140). In some cases, a smart collar 110 may be capable of communicating both directly or indirectly with the network 140. In such cases, whether the smart collar 110 communicates directly or indirectly may be based on one or more conditions (e.g., channel conditions, a power savings mode, a state of a battery of the smart collar 110, and/or availability of a direct connection). For example, when the smart collar 110 is in a power savings mode or the battery power is below a threshold level, the smart collar 110 may communicate indirectly with the network 140, which may utilize less power than communicating directly with the network 140.

For such direct or indirect connectivity with the network 140, the smart collar 110 may include one or more radios (e.g., transceivers 808) that support one or more radio access technologies (RATs) or other type of wireless technologies (e.g., audio or light communications that may not need an antenna). In some cases, a particular RAT or RATs supported may be determined based on cost considerations. Examples of such RATs include, but are not limited to Bluetooth, Bluetooth low energy (LE), Zigbee, WiFi (or other type of wireless local area network-WLAN), or Cellular (or other type of wireless wide area network-WAN). Thus, a wide variety of options exist for providing a gateway to connect the smart collar 110 to the network 140 directly or indirectly. In some cases, the smart collar may communicate using (piggyback on) one or more existing gateways, such as Amazon Echo, smart televisions with radios and networking, home automation and/or security systems, or using a dedicated Gateway (such as 2net).

In general, any suitable type of gateway or direct communications (or other type connectivity) may be supported to allow the smart collar 110 to communicate information via the network 140. As will be described in greater detail below, in some cases, two-way communication may be used between the smart collar 110 and the cloud. For example, data may flow from the smart collar 110 to the cloud (for example, in an event driven manner or in a request/response manner) and data may also flow from the cloud to the smart collar 110 (e.g., configuration information, notifications, or other type information).

In some embodiments, the smart collar 110 may be selectively configured via the cloud. In some embodiments, the smart collar 110 may request the patient to approve any configuration. In some embodiments, the smart collar 110 may also provide the patient options to select what kinds of alerts to receive or what kinds of information to provide over the cloud to devices of the network 140.

As noted above, in some cases, the smart collar 110 may provide functionality even during times when there is no connection to the cloud (times of Non-Connectivity). In such cases, the smart collar may be configured to collect and store data until a connection is gained. In some cases, once activated and configured, data collected by the collar may be encrypted and stored locally on the collar. Such data may be uploaded to the cloud opportunistically (e.g., when connectivity becomes available). In some cases, data may be erased from collar once cloud confirms correct receipt of that data (e.g., via an acknowledgement message).

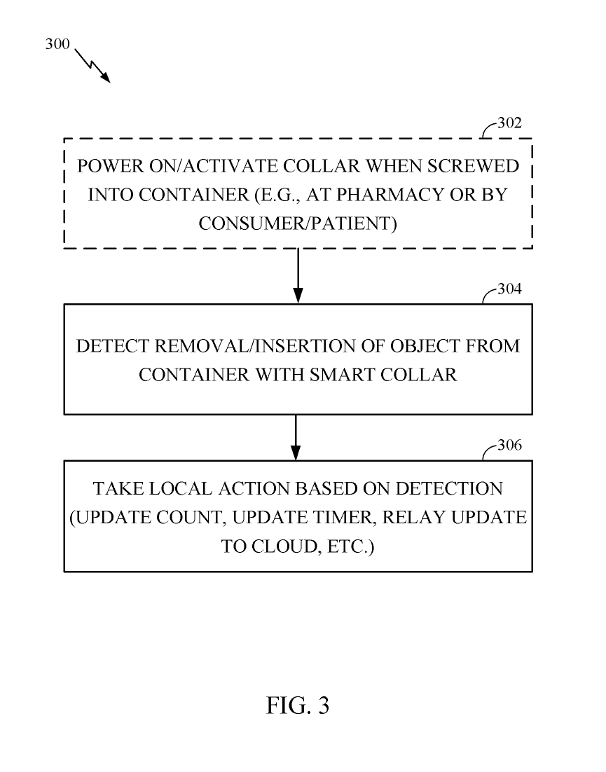

FIG. 3 illustrates example operations 300 for monitoring object removal (and/or insertion) from a container, in accordance with certain aspects of the present disclosure. The operations 300 may be performed, for example, via one or more components (e.g., sensors and processors) of the smart collar 110.

As illustrated at 302, in some cases, the smart collar may be powered on or activated when installed. For example, the smart collar may be powered on or activated when screwed into a pill bottle at a pharmacy or by the patient. In some cases, a smart collar may be activated during assembly/manufacture of a pre-packaged container (e.g., containing a fixed dosage of pills or other type medicine). In some cases, a bottle may be “provisioned” with information related to a particular patient medication regimen, type of medicine (e.g., pill type/size) as well as various other type of configuration information. At 304, the smart collar detects removal of an object from (and/or addition of an object to) the container. At 306, the smart collar takes action based on the detection.

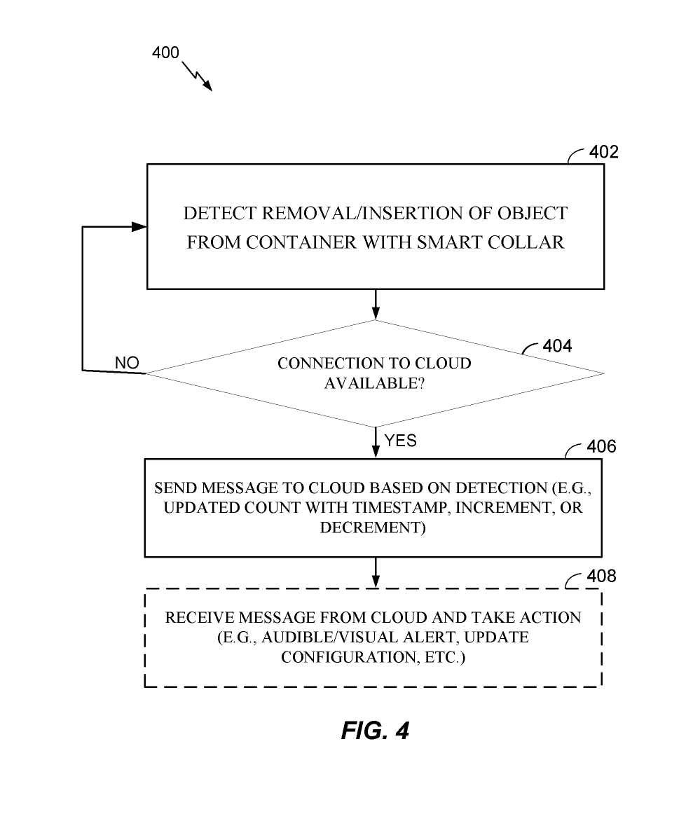

As described above and illustrated in operations 400 of FIG. 4, in some cases, the action taken may depend on current connectivity to the cloud. At 402, the smart collar detects removal of an object from (or addition of an object to) a container. If a connection to the cloud is not available, as determined at 404, for example, the smart collar may update and maintain a pill count locally and continue to detect a change in pill count.

Once a connection is available, at 406, the smart collar may send a message to the cloud. For example, the message may indicate an updated count, a change (increment or decrement) in pill count and may include a timestamp or an indication of a time period over which the count changed. As noted above, the message may be sent directly to the cloud or indirectly (e.g., via a smartphone).

Optionally, at 408, the smart collar may receive a message from the cloud and take action accordingly. The message, for example, may be generated in response to the adherence information sent (at 406). As an example, the message may be a reminder sent via the smartphone app described above (or sent directly to the smart collar). In some cases, the collar may have a mechanism to provide audible alerts (e.g., a speaker or buzzer) and/or visual alerts (e.g., via an LED or small display). As another example, the message may simply acknowledge the cloud received the message sent at 406 (e.g., and the smart collar may delete locally stored data in response to the acknowledgement).

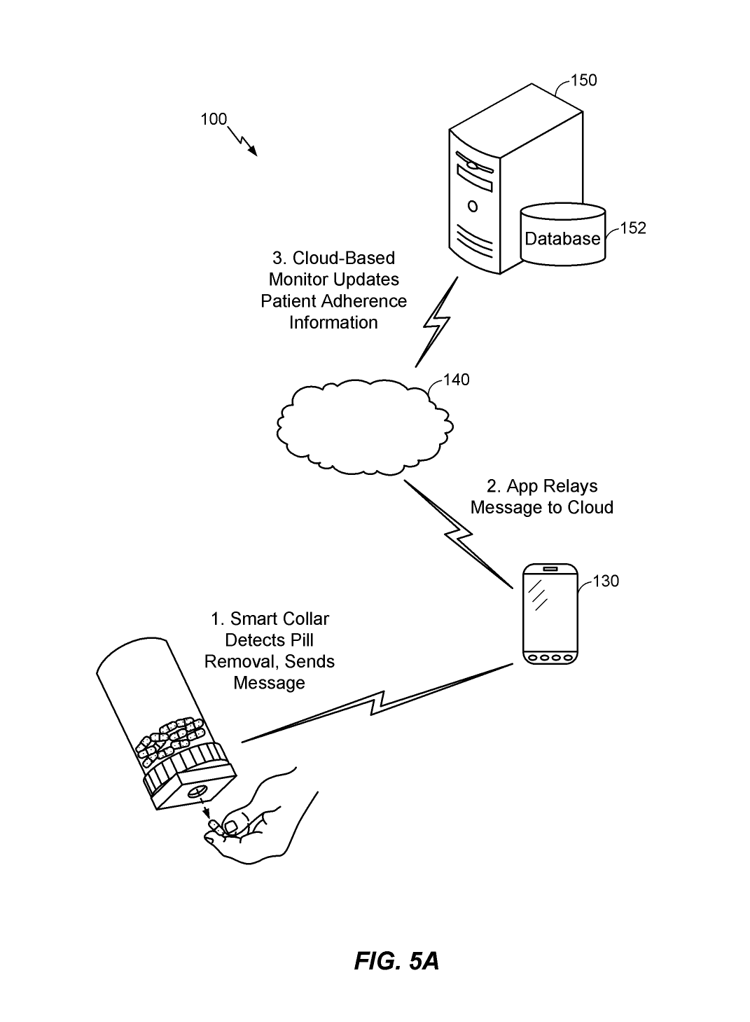

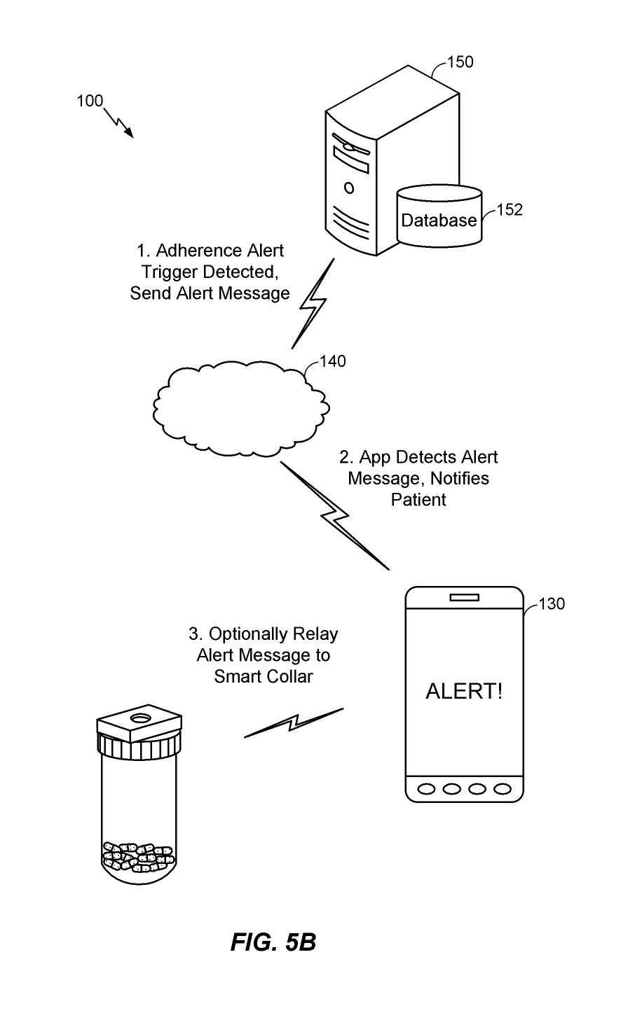

FIG. 5A illustrates an example sequence of operations for updating adherence information via a container with a smart collar, in accordance with certain aspects of the present disclosure. As illustrated, the smart collar may first detect pill removal and send a message to the cloud, relayed through a smartphone in this example. The cloud-based monitoring (e.g., at server 150) may then update patient adherence information accordingly (e.g., in database 152).

As illustrated in FIG. 5B, in some cases, the cloud-based monitoring may generate an alert message (or other notification) in response to some trigger event. For example, a simple reminder may be sent periodically if the server does not receive an update indicating a patient has taken medicine according to a prescribed regimen. As another example, the trigger event may be caused if a timer has expired indicating the patient is not adhering to a prescribed regimen (e.g., taking too few or too many pills). In the illustrated example, the smartphone app may relay such an alert message to the smart collar (e.g., which may provide an audible or visual alert to a patient). In some cases, providers (e.g., doctors) may actually be able to adapt a medication regimen based on the adherence information (and possibly based on other information, such as bio-informatics obtained for the patient).

While the illustrated examples of FIGS. 5A and 5B shows indirect communication between the smart collar 110 and the cloud (via wireless device 140), a similar sequence of messages could be exchanged directly between the cloud and smart collar (e.g., eliminating step 2 in both cases).

### An Example Smart Collar

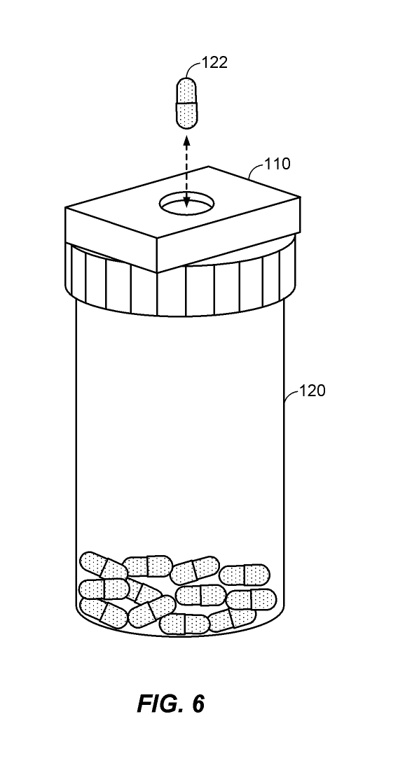

FIG. 6 illustrates an example container with a smart collar for monitoring removal and/or insertion of items from the container, in accordance with certain aspects of the present disclosure. As illustrated, the smart collar may seamless integrate with existing types of pill bottles.

In some cases, the smart collar may be automatically activated (turned on and begin monitoring/reporting) when attached to bottle. This activation may take place, for example, when configured with regimen and/or medicine information. In some cases, the collar may be designed to make it very difficult to remove once it is attached (e.g., via a mechanical device). This may allow for “lifetime tracking” of the container and/or its contents. In some cases, other sensor information (besides removal for adherence) may be monitored (e.g., over the lifetime). Examples of such sensor information may include humidity, temperature, impact, or even location (e.g., via GPS or other means).

In some cases, a sensor may detect removal and trigger an alarm and/or sending of a message. As noted above, the collar may record and report removal of pills from container 120. In some cases, the collar may include some type of visual tamper detection (e.g., such as the “twist off” caps with break-off rings sometimes used on certain types of bottles). Various other types of container (e.g., bottle) security may also be implemented, for example, for tamper detection as well as counterfeit detection (e.g., using a secure ID).

This monitoring functionality may be combined with certain “user facing” communications (e.g., alerts or other type notification). Such alerts/notification may be provided by any suitable techniques, for example, via the smartphone app or via a contact person/means identified by the platform. In some cases, the smart collar may include a mechanism for alerts, such as a buzzer or speaker for audible alerts and/or an LED or display for visual alerts.

In some cases, a collar may be provisioned with information that uniquely identifies the drug (e.g., drug type, manufacturer, date, location, and an authentication ID)—this may be in conjunction with the lifetime tracking mentioned above and may also be used to verify origin and authenticity of drug. Such provisioning may also include medication use guide/information.

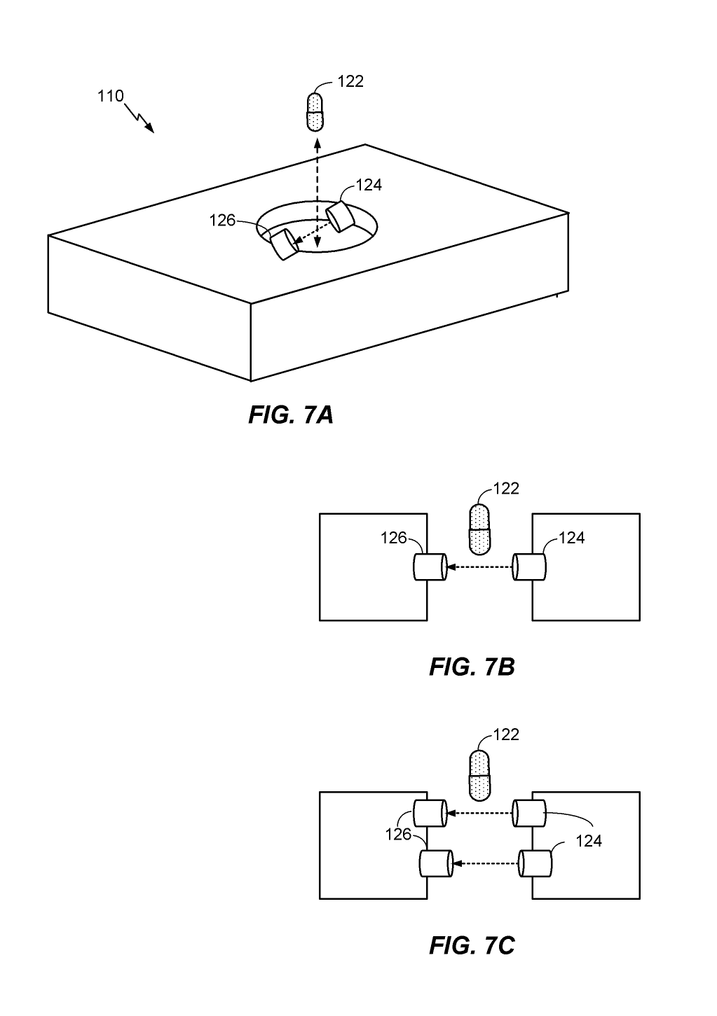

FIG. 7A-7C illustrates example smart collars with one or more sensors to detect travel of an object (such as a pill) from an opening of the smart collar, in accordance with certain aspects of the present disclosure. As noted above, for relatively low cost, a sensor arrangement may allow a smart collar to provide relatively accurate results (e.g., a pill count with “per-pill” resolution).

As illustrated in FIGS. 7A and 7B, one type of sensor arrangement may be a photo interrupter formed by an emitter 124 (e.g., LED) and a detector 126 (e.g., a phototransistor). In such a case, passage of a pill 122 (or other object) may be detected as an interruption in light (emitted from emitter 124) detected at detector 126. As illustrated in FIG. 7B, the emitter/detector pair may be located at an opening through which the object passes. In some cases, the removal (or insertion) of multiple pills may be detected by monitoring the signal (e.g., and how long the path is interrupted based on pill size).

As illustrated in FIG. 7C, in some cases, multiple photointerrupters may be offset (e.g., vertically or horizontally). The offset may help determine direction (to discern removal from insertion) of movement of the object based on which photo interrupter path is interrupted first. In some cases, multiple photointerrupters may also be offset at different angles about the opening, which may help provide results that are more accurate. More elaborate sensing schemes may also be used, albeit in some cases with an increased cost. In some embodiments, the multiple photo interrupters may comprise a single light emitter 124 and multiple detectors 126.

While the examples shown in FIGS. 7A-7C utilize a photo-interrupter, various other approaches may be used for pill counting. For example, various other types of optical sensors may be used, such as a one or more cameras or other imaging methods, placed in multiple topologies (centralized or distributed).

In some cases, rather than a photo interrupter (also known as a transmissive optical sensor), a reflective optical sensor may be used. Transmissive optical sensors detect an object when a light beam is interrupted. Reflective optical sensors measure the amount of light reflected by a passing object. Other types of mechanical or proximity sensors may also be used. In general, any suitable mechanism capable of detecting addition or removal of objects from a container (e.g., pills from a bottle) may be utilized.

In some cases, one type of sensor that may be included is an accelerometer. In such cases, the accelerometer may be used for various functions including: power management, discerning removal from insertion, and collecting information about bottle motion. In some cases, sensor fusion may be performed to improve event detection accuracy. In some embodiments, the accelerometer may provide an indication of an orientation of the smart collar 110 or any other motion. Accordingly, the accelerometer may indicate when the container 120 is tilted or rotated in a particular direction to dispense medication or when it is in an upright (or non-dispensing) position. Thus, the accelerometer may be used to discern when medication is being dispensed or when medication is being inserted. For example, when the accelerometer indicates a tilted position and the photodetector sensor sees passage of medication, the smart collar 110 (via the processor 802) may determine that medication is being dispensed. When the accelerometer indicates a non-tilted position and the photodetector sensor sees passage of medication, the smart collar 110 (via the processor 802) may determine that medication is being inserted.

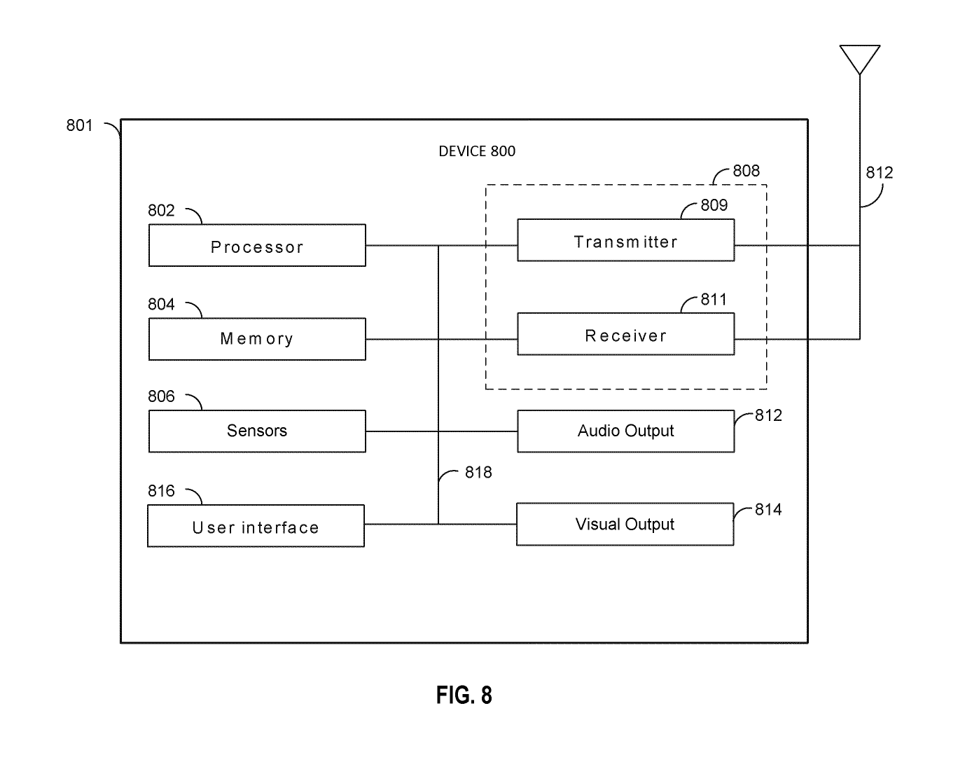

FIG. 8 illustrates an example schematic diagram of a device, in accordance with aspects of the present disclosure. FIG. 8 illustrates various components that may be utilized in a device 800 of the network 140 e.g., the smart collar 110, the smartphone 130, or the server 150 described in connection with FIG. 1) that may be employed within the system 100 of FIG. 1. The device 800 is an example of a device that can be configured to implement the various methods described herein. In some embodiments, the device 800 may be the smart collar 110 or the server 150 or the smartphone 130. With respect to the description of FIG. 8 herein, some of the item numbers may refer to the so-numbered aspects described above in connection with FIG. 1.

As illustrated, the smart collar may include a processor 802, which controls operation of the device. The processor 802 may also be referred to as a central processing unit (CPU) or hardware processor. Memory 804, which may include both read-only memory (ROM) and random access memory (RAM), provides instructions and data to the processor 802. A portion of the memory 804 may also include non-volatile random access memory (NVRAM). The processor 802 typically performs logical and arithmetic operations based on program instructions stored within the memory 804. The instructions in the memory 804 may be executable to implement the methods described herein. Furthermore, the device 800 may utilize the memory 804 to store information about other devices on the network to enable the use of certain methods described below, e.g., storing identifiers for particular smartphones 130 or servers 150 and/or characteristics for the smartphone 130 or servers 150 on the network 140. The device 800 may then utilize the processor 802 in connection with the memory 804 to analyze the dispensing and inserting events, medication regimen data, and time to determine and/or identify adherence or non-adherence conditions of the medication regimen. In some implementations, the processor 802 may include a clock (or the device 800 may include a standalone clock, not shown in this figure). The clock may be used to allow the device 800 to track a time (e.g., a time at which an object is dispensed from or inserted into the container 120).

The smart collar 110 may also include memory 804, for example, In some embodiments, the memory 804 may store instructions executable by processor 802, to store configuration information, adherence information (e.g., pill count), or other type of information. As noted above, in some cases, memory 804 may include information regarding medication (dosage, precautions, and the like) as an alternative (or in addition) to information typically provided in print form.

The processor 802 may comprise or be a component of a processing system implemented with one or more processors. The one or more processors may be implemented with any combination of general-purpose microprocessors, microcontrollers, digital signal processors (DSPs), field programmable gate array (FPGAs), programmable logic devices (PLDs), controllers, state machines, gated logic, discrete hardware components, dedicated hardware finite state machines, or any other suitable entities that can perform calculations or other manipulations of information.

The processing system may also include non-transitory machine-readable media for storing software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing system to perform the various functions described herein. The processor 802 may further comprise a packet generator to generate packets for controlling operation and data communication.

The device 800 may include a transmitter (transmitter circuit) 809 and a receiver (receiver circuit) 811 to allow transmission and reception of data between the device 800 and a remote location and/or device. The transmitter 809 and the receiver 811 may be combined into a transceiver 808. An antenna 812 (or multiple antennas) may be electrically coupled to the transceiver 808. The device 800 may also include (not shown) multiple transmitters, multiple receivers, multiple transceivers, and/or multiple antennas, which may be utilized during multiple-input multiple-output (MIMO) communications, for example. In some embodiments, each of the multiple antennas may be dedicated for the transmission and/or reception of LTE-U, LTE-D, MuLTEfire, and/or WLAN communications. The wireless device may be covered by a housing unit 801.

In some embodiments, the device 800 may also comprises a modem or other communication means (not shown in this figure). For example, the additional communication means may enable the device 800 to send, receive, and process communications, for example via a particular communication medium or protocol. As noted above, the communication means or the transceiver 808 may include any type of wireless interface suitable to allow the device 800 to communicate directly or indirectly, via one or more antennas with the network 140 or any other network.

The device 800 may also include sensors 806 that may be used in an effort to detect and quantify various conditions that the device experiences. For example, in the smart collar 110, the sensors 806 may be used to detect dispensing or inserting of medication into and from the container 120. In some embodiments, the processor 802 may receive input from the sensors 806 (e.g., photo interrupters described above) to detect removal/insertion of pills. In addition, the sensors 806 may include sensors for monitoring various other parameters (e.g., temperature, humidity, location, and the like).

The device 800 may further comprise a user interface 816 in some aspects. The user interface 816 may comprise a keypad, a microphone, a speaker, and/or a display. The user interface 816 may include any element or component that conveys information to a user of the device 800 and/or receives input from the user.

Various components of the device 800 may be coupled together by a bus system 818. The bus system 818 may include a data bus, for example, as well as a power bus, a control signal bus, and a status signal bus in addition to the data bus. Those of skill in the art will appreciate various components of the device 800 may be coupled together or accept or provide inputs to each other using some other mechanism.

Although a number of separate components are illustrated in FIG. 8, those of skill in the art will recognize that one or more of these components may be implemented not only with respect to the functionality described above, but also to implement the functionality described above with respect to other components. For example, the processor 802 may be used to implement not only the functionality described above with respect to the processor 802, but also to implement the functionality described above with respect to the sensor 806 and/or the user interface 816. Each of the components illustrated in FIG. 8 may be implemented using a plurality of separate elements.

As noted above, the device 800 may comprise the smart collar 110 or the smartphone 130, and may be used to transmit and/or receive communications over licensed or unlicensed spectrums and/or exchange communication with each other and/or other devices on the network 140.

The processor 802 may be configured to carry out operations described above. For example, when the device 800 implements the smart collar 110, the processor 802 may help activate the smart collar 110 upon detecting its deployment (e.g., installation on a bottle) to begin monitoring of patient adherence.

As noted above, the smart collar 110 may also include mechanisms to provide audible and/or visual alerts. For example, the smart collar 110 may include a buzzer (or speaker) 812 to provide audible alerts and/or an LED (or display) 814 to provide visual alerts.

In some cases, processor 802 may take measures to conserve power, for example, so the smart collar can operate off a single battery for an extended period (e.g., years). To that end, the processor 802 may implement any combination of one or more power optimization techniques to conserve power while monitoring sensors and/or transmitting messages.

In some embodiments, the smart collar 110 may be configured to operate in an isolated manner (e.g., without communications to the cloud network described herein or with minimal such communications). For example, in some embodiments, the smart collar 110 may be configured to perform active adherence, or closed loop monitoring, of the patient adhering to the medication regimen. The active adherence or closed loop monitoring may include determining, without communicating with the smartphone 130 or the server 150, that the patient is adhering to the medication regimen.

When the smart collar 110 performs the active adherence or closed loop monitoring itself, the smart collar 110 may be configured to monitor dispensing and inserting events and compare those events with the medication regimen of the patient. For example, the smart collar 110 may store the events in its memory (e.g., memory 804) and compare the times of the events with the expected times as indicated in the medication regimen. Accordingly, the smart collar 110 may actively monitor the patient's adherence to the medication regimen in a closed loop without requiring intervention or communication with any external devices. For example, the medication regimen may be stored in the memory of the smart collar 110 with the details of the medication contained within the container 120. Accordingly, the smart collar 110 may include all information it needs to perform the adherence monitoring.

In some embodiments, the active adherence may involve monitoring the dispensing of medication from the container 120 (e.g., number of pills 122 and time at which they are dispensed) via the smart collar 110 (e.g., via the processor 802). The processor 802 may further correlate the dispensing (or inserting) information with the patient's medication regimen. Correlating the dispensing information may comprise comparing the dispensing information (e.g., time, quantity of medication dispensed, etc.) with the medication regimen to see if they match or if there are any discrepancies. In some embodiments, the smart collar 110 may generate alerts to the patient to remind of the need to dispense the medication or to indicate that a dispensing period was missed. In some embodiments, the smart collar 110 may generate alerts to the patient to indicate that too much medication was dispensed and that some of the dispensed medication should be reinserted into the container 120 via the smart collar 110.

The smart collar 110 may customize alerts based on a determination that one or more dispensing events (e.g., scheduled medication use times) were missed. In some embodiments, the smart collar 110 may include varying levels of alerts and may progress to different levels based on a severity of the missed dispensing event(s). The varying levels of alerts may progressively increase in an amount of alert or in the level of attention the alert garners. For example, a low severity level alert may simply include a text message or warning. A higher severity level alert may include audible or visual alerts. Even higher severity level alerts may include calls to people associated with the patient.

Thus, if the patient misses a single dispensing event, then the smart collar 110 may determine to generate a text reminder. However, if the patient misses a consecutive, subsequent dispensing event, the smart collar 110 may advance to a different level of alert (e.g., an audible alert or visual (e.g., colored lights) alert). If many days are of events are missed, the smart collar 110 may advance to communicating with people associated with the patient, healthcare providers, or emergency response personnel.

In some embodiments, the smart collar 110 may be configured to encrypt and record each dispensing event (and any insertion events). In such embodiments, the patient's smartphone 130 may be configured to communicate the encrypted and/or recorded information to a device on the cloud (e.g., the server 150 that tracks the patient's adherence to the medication regimen). In some embodiments, by encrypting the dispensing and insertion events at the smart collar 110, confidentiality of the patient's healthcare information may be maximized or improved over systems that encrypt the information at the smartphone 130 or similar device. In some embodiments, the smart collar 110 may be configured to record a specified number of events in a local memory (e.g., the memory 804). This ability to record the events in local memory 804 may allow the smart collar 110 to be synchronized on a less frequent basis than smart collars 110 without local memory 804. Such less frequent synchronization may promote power savings for the smart collar 110 and reduced transmission of healthcare information. In some embodiments, the smart collar 110 may be configured to communicate with the smartphone 130 and the devices in the cloud with minimum latency. The minimum latency may provide for more prompt monitoring when a patient took or missed their pills 122, how many pills 122 were dispensed or missed, and compare the monitored events to medication regimen (e.g., schedule) they have been given by their healthcare provider. Accordingly, the cloud device monitoring the events may act on discrepancies or non-adherence events.

In some embodiment, when the processor 802 is encrypting the information for communication to the smartphone 130 or the server 150, the processor 802 may first select an encryption key from a library of encryption keys. The library of encryption keys may be stored in the memory 804. In some embodiments, each device with which the smart collar 110 communicates may have its own encryption key. In some embodiments, the encryption key may be rotating to minimize the likelihood of the encryption key being guessed by an adverse party. In some embodiments, different devices of the network 140 may utilize different encryption keys. For example, a pharmacy communicating over the network 140 may use a first encryption key while a healthcare provider communicating over the network 140 may use a second encryption key. Similarly, a third encryption key may be used by the patient's family or home network. Accordingly, the processor 802 may select the encryption key to user from the library of encryption keys based on the destination of information being communicated. In some embodiments, the processor 802 may include the encryption key in the encrypted information for ease decryption.

Similarly, in some embodiments, the processor 802 may be configured to decrypt information that is received via the network 140. For example, the smart collar 110 may receive an encrypted communication regarding the medication in the container 120. Accordingly, the smart collar 110 may identify a proper decryption key to use (e.g., from the library of encryption keys) to properly decrypt the received communication.

In some embodiments, event reporting (e.g., the dispensing or inserting of medication as detected by the smart collar 110) may be performed in a timely but power efficient manner. For example, the smart collar 110 may determine when it is connected to one or more cloud devices (e.g., server 150) and coordinate reporting of events to the server 150 based on the connectivity status of the smart collar 110. For example, when the smart collar 110 determines that it is not connected to one or more cloud devices (e.g., server 150), the smart collar 110 may hold reporting of events in order to reduce the transmission of data during periods when the smart collar 110 is not connected to the server 150. In some embodiments, the smart collar 110 may be configured to implement a back-off procedure or other communication scheduling procedure. In some embodiments, when the smart collar 110 detects an event, the smart collar 110 implements a power efficient communication algorithm to connect to the server 150. For example, the algorithm may include the back-off procedure and may include storing of multiple event information for communication to the server 150 once the communication to the server 150 is re-established.

As described above, certain aspects of the present disclosure provide a cloud-based platform that may utilize a smart collar to monitor patient adherence to a medication regimen. The smart collar may provide a relatively low-cost mechanism that seamlessly integrates in existing containers (e.g., standard pill bottles of various sizes) and provides accurate monitoring of medication consumption. This information may be provided to a cloud-based monitoring system to help monitor and promote patient adherence to a regimen, which may lead to improved results, potentially increasing wellness and saving both cost and lives.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members as well as combinations that may multiples of the same element.

The various operations of methods described above may be performed by any suitable means capable of performing the corresponding functions. The means may include any suitable combination of hardware, software, or both. Examples of such hardware include any suitable circuit or circuitry, such as a processor (e.g., a general-purpose processor, field programmable gate array (FPGA), or application specific integrated circuit (ASIC).

Portions implemented in software may include code or instructions on a computer-readable medium. As used herein, the term software generally refers to any combination of instructions, data, or both, and may include firmware. A computer-readable storage medium may be integral to a processor of coupled to a processor such that the processor can read information from, and write information to, the storage medium. Examples of machine-readable storage media may include, for example, one or more of: any type of Random Access Memory (RAM), Read Only Memory (ROM), or any other suitable storage medium.

It is to be understood that the claims are not limited to the precise configuration and components illustrated above. Various modifications, changes and variations may be made in the arrangement, operation and details of the methods and apparatus described above without departing from the scope of the claims.

The various operations of methods described above may be performed by any suitable means capable of performing the operations, such as various hardware and/or software component(s), circuits, and/or module(s). Generally, any operations illustrated in the Figures may be performed by corresponding functional means capable of performing the operations.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logical blocks, modules, circuits, and method steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the implementations.

The various illustrative blocks, modules, and circuits described in connection with the implementations disclosed herein may be implemented or performed with a general purpose hardware processor, a Digital Signal Processor (DSP), an Application Specified Integrated Circuit (ASIC), a Field Programmable Gate Array (FPGA) or other programmable logic device, discrete gate or transistor logic, discrete hardware components, or any combination thereof designed to perform the functions described herein. A general-purpose hardware processor may be a microprocessor, but in the alternative, the hardware processor may be any conventional processor, controller, microcontroller, or state machine. A hardware processor may also be implemented as a combination of computing devices, e.g., a combination of a DSP and a microprocessor, a plurality of microprocessors, one or more microprocessors in conjunction with a DSP core, or any other such configuration.

The steps of a method and functions described in connection with the implementations disclosed herein may be embodied directly in hardware, in a software module executed by a hardware processor, or in a combination of the two. If implemented in software, the functions may be stored on or transmitted as one or more instructions or code on a tangible, non-transitory computer readable medium. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD ROM, or any other form of storage medium known in the art. A storage medium is coupled to the hardware processor such that the hardware processor can read information from, and write information to, the storage medium. In the alternative, the storage medium may be integral to the hardware processor. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer readable media. The hardware processor and the storage medium may reside in an ASIC.

For purposes of summarizing the disclosure, certain aspects, advantages and novel features have been described herein. It is to be understood that not necessarily all such advantages may be achieved in accordance with any particular implementation. Thus, the invention may be embodied or carried out in a manner that achieves or optimizes one advantage or group of advantages as taught herein without necessarily achieving other advantages as may be taught or suggested herein.

Various modifications of the above-described implementations will be readily apparent, and the generic principles defined herein may be applied to other implementations without departing from the spirit or scope of the application. Thus, the present application is not intended to be limited to the implementations shown herein but is to be accorded the widest scope consistent with the principles and novel features disclosed herein.

What is claimed is:

1. An apparatus for tracking movement of an object within a container, the apparatus comprising:
a sensor circuit configured to:
detect at least one of a removal of the object from or insertion of the object into a container, and
generate a signal in response to detecting the removal of the object from or insertion of the object into the container;
a hardware processor configured to:
determine a time at which the object was removed from or inserted into the container based on the signal generated by the sensor circuit,
generate a message indicating that the object was removed from or inserted into the container at the determined time,
select an encryption key from a memory circuit storing a library of encryption keys, and
encrypt the generated message for transmission to a monitoring device according to the selected encryption key; and
a transmitter circuit configured to wirelessly transmit the encrypted message to the monitoring device.

2. The apparatus of claim 1, wherein the signal generated by the sensor circuit is indicative of a quantity of objects removed from or inserted into the container and wherein the message generated by the hardware processor indicates the quantity of objects removed from or inserted into the container.

3. The apparatus of claim 1, further comprising a housing configured to house the sensor circuit, the hardware processor, and the transmitter circuit, wherein the housing includes an opening that passes through the housing and wherein the housing is positioned at a mouth of the container.

4. The apparatus of claim 1, wherein the memory circuit is configured to store a medication regimen, the medication regimen including a schedule according to which the object is to be removed from the container, wherein the hardware processor is further configured to compare the time at which the object was removed from or inserted into the container with the schedule.

5. The apparatus of claim 1, wherein the sensor circuit comprises a light emitting diode (LED) and a photodetector and wherein the sensor circuit generates the signal when the object removed from or inserted into the container interrupts a path of light between the LED and the photodetector.

6. The apparatus of claim 5, wherein the sensor circuit further comprises an accelerometer configured to discern removal from or insertion into the container of the object.

7. The apparatus of claim 5, wherein the sensor circuit comprises at least a first photodetector and a second photodetector and wherein the first and second photodetectors are offset from each other vertically or horizontally.

8. The apparatus of claim 7, wherein the hardware processor is configured to process signals from the first and second photodetectors before updating a count of the objects in the container.

9. The apparatus of claim 5, wherein the hardware processor is configured to update a change in a count of the objects in the container if the signal indicating the object has interrupted the path of light between the LED and the photodetector for at least a threshold duration.

10. The apparatus of claim 9, wherein the threshold duration is based, at least in part, on one or more dimensions of the objects.

11. A method for tracking movement of an object within a container, the method comprising:
detecting at least one of a removal of the object from or insertion of the object into a container;
generating a signal in response to detecting the removal of the object from or insertion of the object into the container;
determining a time at which the object was removed from or inserted into the container based on the signal generated by the sensor circuit;
generating a message indicating that the object was removed from or inserted into the container at the determined time;
selecting an encryption key from a memory circuit storing a library of encryption keys;
encrypting the generated message for transmission to a monitoring device according to the selected encryption key; and
wirelessly transmitting the encrypted message to the monitoring device.

12. The method of claim 11, wherein the generated signal is indicative of a quantity of objects removed from or inserted into the container and wherein the message generated indicates the quantity of objects removed from or inserted into the container.

13. The method of claim 11, further comprising a housing configured to house a sensor circuit, a hardware processor, and a transmitter circuit, wherein the housing includes an opening that passes through the housing and wherein the housing is positioned at a mouth of the container.

14. The method of claim 11, further comprising:
storing a medication regimen in the memory circuit, the medication regimen including a schedule according to which the object is to be removed from the container; and
comparing the time at which the object was removed from or inserted into the container with the schedule.

15. The method of claim 11, further comprising generating the signal when the object removed from or inserted into the container interrupts a path of light between a light emitting diode (LED) and a photodetector.

16. The method of claim 15, further comprising discerning removal of the object from or insertion of the object into the container using an accelerometer.

17. The method of claim 15, wherein the photodetector comprises at least a first photodetector and a second photodetector and wherein the first and second photodetectors are offset from each other vertically or horizontally.

18. The method of claim 17, further comprising processing signals from the first and second photodetectors before updating a count of the objects in the container.

19. An apparatus for tracking movement of an object within a container, the apparatus comprising:
means for detecting at least one of a removal of the object from or insertion of the object into a container;
means for generating a signal in response to detecting the removal of the object from or insertion of the object into the container;
means for determining a time at which the object was removed from or inserted into the container based on the signal generated by the sensor circuit;
means for generating a message indicating that the object was removed from or inserted into the container at the determined time;
means for selecting an encryption key from a memory circuit storing a library of encryption keys;
means for encrypting the generated message for transmission to a monitoring device according to the selected encryption key; and
means for wirelessly transmitting the encrypted message to the monitoring device.

20. The apparatus of claim 19, wherein:
the means for detecting comprises a sensor circuit,
the means for generating a signal comprises the sensor circuit,
the means for determining a time comprises a hardware processor,
the means for generating a message comprises the hardware processor,
the means for selecting an encryption key comprises the hardware processor,
the means for encrypting the generated message comprises the hardware processor, and
the means for wirelessly transmitting the encrypted message comprises a transmitter circuit.
